(19) United States
(12) Patent Application Publication
(10) Pub. No.: US 2023/0355542 A1
(43) Pub. Date: Nov. 9, 2023

(54) DRUG-ELUTING DEVICE WITH GENETIC AND CHEMICAL THERAPEUTICS FOR TREATING OR PREVENTING VASCULAR ACCESS DYSFUNCTIONS

(71) Applicant: Yasin Oduk, Hoover, AL (US)

(72) Inventor: Yasin Oduk, Hoover, AL (US)

(21) Appl. No.: 18/312,067

(22) Filed: May 4, 2023

Related U.S. Application Data
(60) Provisional application No. 63/364,145, filed on May 4, 2022.

(51) Int. Cl. A61K 9/70; A61M 1/36; A61K 47/69; A61K 31/7105; A61K 31/565; A61P 13/12

(52) U.S. Cl. CPC A61K 9/7007; A61M 1/3655; A61K 47/6937; A61K 31/7105; A61K 31/565; A61P 13/12; A61B 2017/00641

(57) ABSTRACT

An implantable drug delivery device includes a biodegradable substrate and polymeric nanoparticles containing a therapeutic agent affixed to the biodegradable substrate. A method of preventing or reversing vascular hemodialysis access dysfunction includes wrapping an anastomosis injury site with the implantable drug delivery device. A method of making the implantable drug delivery device includes forming a substrate; forming nanoparticles containing a therapeutic agent by encapsulation and/or from an emulsion; and applying the nanoparticles to the substrate.

### CROSS-REFERENCE TO RELATED APPLICATION

This application claims the benefit of priority of U.S. provisional application No. 63/364,145, filed May 4, 2022, the contents of which are herein incorporated by reference.

### BACKGROUND OF THE INVENTION

The present invention relates to vascular access dysfunctions and, more particularly, to a drug-eluting device with genetic and chemical therapeutics for treating or preventing vascular access dysfunctions. End-Stage Renal Disease (ESRD) is the final stage of chronic kidney disease (CKD), the gradual decrease of kidney function over time. In 2018, approximately 131k new cases of End Stage Renal Disease (ESRD) were reported and 70% of patients with ESRD dependent on hemodialysis three times a week. According to the Center for Disease Control and Prevention (CDC) over 700,000 patients lived with ESRD in the US. Vascular access is a lifeline for patients on hemodialysis. At present, a recent study from the United States has reported that $2.8 billion US dollars, which is ˜12% of the entire Medicare budget, are spent on dialysis vascular access services [1].

Arteriovenous fistulas (AVFs), i.e., arteriovenous dilatation that makes it suitable to perform dialysis, are the preferred vascular access means due to the lowest complication rate and greater blood flow compared to other currently available means. In the current practice environment, the fistula first initiative emphasizes the creation of AVF over arteriovenous graft (AVG) or catheter placement [2]. Once the access is created, blood flows from the high-pressure artery to, and through, the low-pressure pressure vein. Over time, the wall of the vein thickens, its diameter enlarges, and it becomes suitable to perform dialysis. It can take up to 6 months for the AVFs to mature[3]. Until fistulas mature, patients have no choice but to undergo dialysis with a catheter, which is a major cause of infection, hospitalization, and death.

But AVF maturation failure remains a significant clinical problem. About 20% to 50% of fistulas created never mature to support dialysis. AVF maturation failure frequently results from venous stenosis at the AVF anastomosis, secondary to poor outward vascular remodeling and excessive neointimal hyperplasia (unwanted tissue in blood vessels), that narrows the AVF lumen. When these fistulae fail to mature, patients are often referred for endovascular management to evaluate and treat lesions that inhibit fistulae maturation. Approximately 50% of fistulas require intervention before successful arteriovenous fistulae use. In some cases, multiple endovascular procedures are applied to aid in the maturation of the fistulas [4]. Furthermore, 27% of fistulae fail and are abandoned within 18 months of creation [5]. Neointimal hyperplasia is responsible for most vascular access dysfunctions [4].

Moreover, vascular access surgeries cause inflammatory responses in the adventitia, resulting in vascular constriction.

Most current therapies have focused on treating vascular access dysfunction by dilating stenosis with angioplasty, mechanical thrombectomy, and stenting rather than preventing it. Those procedures are far from a permanent solution. Instead, they cause shear stress to the vessels and result in re-stenosis [6]. To date, there are no effective therapies to prevent AVF or AVG maturation failure. Recent perivascular and mechanical approaches to prevent AV fistula failure have not received food and Drug Administration (FDA) approval and they are effective for a limited time since they focus on singular problems in AV fistula failure. Moreover, patients are inflammation prone (30-50%) and have major preexisting vascular pathologies (venous intimal hyperplasia, arterial calcification, and arterial intimal hyperplasia) [7],[8],[9],[10],[11],[12]. Therapies must address multiple factors of AV fistula failure and be applicable to the diverse hemodialysis patient population.

There is currently no product available on the market that prevents vascular access dysfunction. One product still in the FDA approval process uses an interior sheet or membrane of type I collagen with an exterior sheet of polytetrafluoroethylene (PTFE) as a skeletal support. The collagen sheet is attached to the PTFE sheet with Sirolimus, a modified antiproliferative drug. The collagen/PTFE product is applied to the outer layer of the anastomosis at the time of surgery and requires a to attach to the target area suture causing further injury. In addition to anastomosis, suturing a product on targeted tissue causes extra stress to the vessels and bleeding. Thus, the unnecessary injury accelerates the inflammation process, which results in the tissue healing longer than expected.

Sirolimus is known as an immunosuppressive drug. It inhibits the growth of vascular smooth muscle cells (SMCs), which is the main reason for neointima formation. But it does not promote reendothelialization to reverse an injured vessel back into a healthy vessel while inhibiting the growth of vascular SMCs.

As can be seen, there is a need for a method or device to reduce vascular access dysfunctions. Further, there is an urgent clinical need to develop therapies to enhance AVF maturation and improve these vascular accesses' short and long-term durability.

### SUMMARY OF THE INVENTION

In one aspect of the present invention, an implantable drug delivery device comprises a biodegradable substrate and polymeric nanoparticles containing a therapeutic agent affixed to the biodegradable substrate.

In another aspect of the present invention, a method of preventing or reversing vascular hemodialysis access dysfunction comprises wrapping an anastomosis injury site with the implantable drug delivery device.

In yet another aspect of the present invention, a method of making the implantable drug delivery device comprises forming a substrate; forming nanoparticles containing a therapeutic agent by encapsulating and/or from an emulsion; and applying the nanoparticles to the substrate.

A general goal of the present invention is to reduce the morbidity associated with hemodialysis vascular access dysfunction. A further goal is to prevent veins from narrowing due to thrombosis, i.e., untreated venous stenosis formed by aggressive neointimal hyperplasia in the AVF surgery site, to keep the portal open and provide consistent vascular access necessary for successful dialysis.

The drug-eluting device may be utilized in other fields or for other purposes. For example: Neointimal hyperplasia is the primary cause of restenosis after percutaneous coronary interventions such as stenting or angioplasty in cardiovascular dysfunctions [13]. The drug delivery device may inhibit neointimal formation, thereby significantly reducing vascular access dysfunction besides restenosis and may substantially reduce the economic cost and health morbidity currently associated with this recalcitrant clinical problem. The device disclosed herein may also be advantageous for Vascular access surgery with respect to arteriovenous fistula (AVF), Vascular access surgery for arteriovenous graft (AVG), carotid endarterectomy surgeries (i.e., plaque removal), drug-eluting coronary stents, drug-eluting urinary stents, and drug-eluting balloon angioplasty. The inventive device is not limited to use in humans and may be used in veterinary medicine.

These and other features, aspects and advantages of the present invention will become better understood with reference to the following drawings, description, and claims.

### BRIEF DESCRIPTION OF THE DRAWINGS

FIG. 1 is a schematic view of nanoparticle-loaded films according to an embodiment of the present invention, shown in use;

FIG. 2 is an alternate schematic view thereof;

FIG. 3 is a graph of E-1 released over time utilizing the nanoparticle-loaded films of FIG. 1;

FIG. 4 is a graph of an amount of E-2 released by day from the films of FIG. 1;

FIG. 5 is a graph of cumulative release of E-1 over time, corresponding to FIG. 3;

FIG. 6 is a graph of cumulative release of E-2 over time, corresponding to FIG. 4;

FIG. 7 is a graph of change in smooth muscle cell proliferation as a function of estrogen-2-loaded nanoparticle concentration;

FIG. 8 is a graph of change in smooth muscle cell proliferation as a function of miR-21-loaded nanoparticle concentration according to an embodiment of the present invention;

FIG. 9 is a graph of a miR-21 content inside nanoparticles;

FIG. 10 is a SEM (scanning electron microscopy) image of estrogen-2 loaded nanoparticles according to an embodiment of the present invention;

FIG. 11 is another SEM image thereof;

FIG. 12 is a photographic view of untreated poly(lactic-co-glycolic acid) PLGA film;

FIG. 13 is a photographic view of a PLGA film with coumarin-6 loaded nanoparticles according to another embodiment of the present invention;

FIG. 14 is a photographic view of the film of FIG. 13 applied to injured mouse carotid artery tissue following ligation;

FIG. 15 is a microphotographic view 1-day post-surgery of a cross section of the mouse carotid artery tissue with applied film of FIG. 14; and

FIG. 16 is a SEM image of microRNA-21-containing nanoparticles according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

As used herein, an “arteriovenous fistula” refers to an abnormal connection between an artery and a vein and “anastomosis” refers to a surgical connection between a vein and an artery. “Neointimal hyperplasia” refers to the presence of unwanted tissue in a blood vessel.

The term “polymeric nanoparticles” refers to submicron colloidal particles in the present disclosure.

The term “peri-vascularly” means from outside of the vascular tissue.

Broadly, one embodiment of the present invention is a drug-eluting device carrying genetic and chemical therapeutics for direct application to an injury site.

For example, the drug-eluting device disclosed herein may be used to treat or prevent vascular access dysfunctions. However, the device may be used with any drug or combination of drugs. Therefore, the device may be applied to colon cancer surgical sites or cancer removal injury sites with wound-healing anti-inflammatories or antibiotics to protect the wound from infection and, in some cases, coagulants to stop bleeding (e.g., for soldiers on the battlefield). As suturing decreases the unity of tissues, brings extra stress, and causes inflammation, a device that provides self-healing abilities without suture increases the chance of healing. Alternatively, chemotherapeutic agents such as paclitaxel, Bleomycin, Doxorubicin, etc., and/or oncogenic agents, such as miRNA 17/92 cluster, may be applied utilizing the inventive drug-eluting device, lessening the effect of systemic chemotherapeutic side effects.

The present drug eluting device prevents or treats neointima formation with a polymer-based film together with nanoparticle therapeutics applied to anastomosis at the same time during vascular access surgery. The inventive drug nanoparticle-coated film may cover 80-90% of the artery and vein to enable the vessel's dilatation.

The key point of this therapy is to inhibit the growth of the vascular smooth muscle cells (SMCs) while promoting re-endothelization of the vessel to reverse the injured vessel back into a healthy vessel. The percentage of the diameter stenosis depends on the reduction of neointima hyperplasia, acceleration of reendothelization, inhibition of thrombosis, and reduction of inflammation.

Nanocarriers have been studied in the pharmaceutical field. Lipid-nanoparticle systems are considered one of the most promising vectors for miRNA delivery. However, polymer-based carriers have shown advantages over lipid-based carriers, such as versatility of structural modifications and protection of unstable microRNA (miRNA) [14].

Polymeric nanoparticles are usually composed of drugs and polymer. Polymeric nanoparticles can promote drug protection and avoid clearance from the body, resulting in a longer half-life. Furthermore, these nanocarriers can reduce drug distribution through healthy tissues, stimulate controlled release, reduce adverse events, increase therapeutic efficiency and safety, overcome physiological barriers, and avoid efflux pumps [15].

The inventive product generally uses FDA-approved biodegradable materials. PLGA and polycaprolactone (PCL)-based polymers are biodegradable and they support the drug-eluting nanoparticle (NP) as a drug source or drug reservoir.

Poly (lactic-co-glycolic acid) (PLGA) is one of the most effective biodegradable polymeric nanoparticles (NPs). The FDA has approved its use in drug delivery systems due to controlled and sustained-release properties, low toxicity, and biocompatibility with tissue and cells. PLGA nanoparticles have exhibited slow-releasing capability for hydrophobic drugs and have also been tested extensively for DNA delivery in gene therapy [16].

Polycaprolactone (PCL) is a promising biodegradable, biocompatible, and non-toxic polymer for developing nanoparticles. Even though PCL has been widely used in the pharmaceutical field, its hydrophobic characteristic leads to fast removal from the body and poor stability of nanoparticles in water. Surface modifications to increase PCL nanoparticle stability may render PCL nanoparticles suitable for use.

Due to their amphiphilic characteristic, poloxamers, also known as Pluronic, have also been used to obtain surface-coated nanoparticles for drug delivery purposes. They are tri-block copolymers composed of hydrophobic and hydrophilic units of propylene oxide (PO) and ethylene oxide (EO), respectively [17].

Gene therapeutics target diseases at the genetic level, using the specificity of complementary binding by nucleic acids to minimize off-target effects. Gene therapy approaches rely heavily on targeted reagents which selectively recognize DNA and ribonucleic acid (RNA) sequences. Innovative gene-activated materials may be produced that encourage the body's cells to produce the proteins needed for vascular tissue repair.

MicroRNAs are a recently discovered class of endogenous, small, noncoding RNAs that regulate about 30% of the encoding genes of the human genome [18], [19], [20] and are involved in the development of many diseases and conditions. For this reason, miRNAs have been proposed and investigated as biomarkers and therapeutics for various diseases like cancer, metabolic diseases, and viral infections [14]. More recently, the roles of miRNAs in cardiovascular biology and disease have received significant attention. MiRNAs may be a new therapeutic target for proliferative vascular diseases such as atherosclerosis, postangioplasty restenosis, transplantation arteriopathy, and stroke [21]. Recently, scientists have focused on developing an efficient targeted delivery system for microRNAs to overcome theft limiting therapeutic aspects [22]. Systemic miRNA delivery for treatment has two types of vectors: viral and nonviral carriers. Nonviral carriers are considered the preferred choice for clinical studies using miRNAs, even though some of them have inefficient miRNA delivery with short efficacy.

The microRNA miR-21 plays essential roles in vascular smooth muscle cell proliferation and apoptosis, cardiac cell growth and death, and cardiac fibroblast functions. In the setting of vascular injury, miR-21 promotes TGF-β1-induced fibroblast differentiation and migration from the adventitia through the media and into the intima, where these cells differentiate into myofibroblasts [23]. Recent studies showed substantial evidence that miR-21 can regulate the differentiation and proliferation of vascular smooth muscle cells after vessel injury. These results suggest that these miRNAs may play a critical role in the development of intimal hyperplasia after vessel injury and may be a potential target of intervention against restenosis [23],[24],[25],[23],[26]. The present disclosure is focused on inhibition or downregulation of highly expressed microRNA-21 in vascular access dysfunctions. The therapy of the present subject matter downregulates mir-21 to prevent fibroblast differentiation into myofibroblasts and to reverse inflammation in the injured vessels to prevent neointima formation from evolving into stenosis. Downregulation of miR-21 has been shown to decrease inflammation, slow neointima formation and stenosis, and thus prevent thrombosis [8].

PLGA nanoparticle-mediated microRNA delivery through a local platform prevents vascular hemodialysis access dysfunctions. For example, both anti-microRNA-21(mir-21) and mir-21 oligonucleotides in a PLGA-mediated nanoparticle delivery system may be used.

The nanoparticle surface may be coated with tissue-specific ligands, such as Ephrin B4, to target the AV anastomosis, which overexpresses Ephrin B2 receptor. Ephrin B4 directly binds to Ephrin B2, downregulating signaling pathways and resulting in cell migration and proliferation. Ephrin B4 also prevents excessive wall thickening and promotes AVF patency [27],[28],[29],[30],[31].

In some embodiments, a dual release local delivery PLGA-based film platform combines ticlopidine to inhibit platelet aggregation and prevent clot formation, as well as anti-mir-21 nanoparticles to prevent multiple pathways of fistula failure. Ticlopidine inhibits platelet aggregation induced by adenosine diphosphate (ADP), blocking platelets, and preventing clot formation [32][33]. Ticlopidine also enables vasodilator responses that are not mediated by endothelial nitric oxide [34].

The PLGA film may contain 230 mg ticlopidine, evenly distributed in the polymer matrix for a steady and controlled drug release due to gradual water penetration into the matrix and due to polymer degradation. Ticlopidine has shown promising results in decreasing fistula failure rates. Dual release of ticlopidine with anti-miR-21 NPs is believed to provide 70-80% less neointima thickness, unrestricted blood flow with increased shear stress, low inflammation markers, and protection from future thrombotic episodes as compared to a control group.

Estrogen is a therapeutic agent active both on vascular smooth muscle cells and endothelial cells where functionally competent estrogen receptors have been identified. Estrogen administration promotes vasodilatation in human and experimental animals [35], in part by stimulating prostacyclin and nitric oxide synthesis and decreasing the production of vasoconstrictor agents such as cyclooxygenase-derived products, reactive oxygen species Angiotensin II, and Endothelin-1. In vitro, estrogen exerts a direct inhibitory effect on smooth muscle by activating potassium efflux and inhibiting calcium influx [36]. In addition, estrogen inhibits vascular smooth muscle cell proliferation. Due to endothelial function improvement, estrogen can inhibit intimal proliferation and accelerate endothelial regeneration [37]. As estrogen does not delay endothelial regrowth, estrogen therapy avoids the risk of late thrombosis.

Estradiol (17Beta-E2) is an estrogen known to inhibit injury-related neointima formation [38], [11] and prevent restenosis after angioplasty, possibly by inhibiting ERK activation in smooth muscle cells and by promoting reendothelialization [40]. Also, Estradiol prevents neointimal thickening after balloon injury and ameliorates the lesions occurring in arteriosclerotic conditions. Systematic E2 vascular treatment may result in a significant decrease in intima/media ratio compared to control drugs [41].

In some embodiments, the present invention comprises sustained released estradiol (E2) nanoparticles from a bio-degradable polymeric wrap to prevent neointimal thickening.

A method of delivering nanoparticle-engineered drugs locally to the blood vessel at the anastomosis includes implanting poly(lactic-co-glycolic acid) (PLGA)-based biodegradable film coated with E2 nanoparticles at the time of the surgical procedure. The films easily and conveniently wrap the injury site without requiring a suture. Perivascular delivery of nanoparticle-loaded drug delivery may achieve high local drug levels while avoiding systemic toxicity. This local approach is ideally suited for use in clinical settings.

The PLGA-based film may be deployed as a perivascular sheath at the time of open vascular surgery. Product size and dosing may be optimized to correlate with the patient's history (vessel size, age, and weight).

Vascular remodeling, which may depend on adventitial angiogenesis, constriction, and the migration of myofibroblasts from the adventitia to the intima, is highly responsive to perivascular drug delivery of appropriate therapeutic agents [42], [43]. Such an outside-in approach allows the delivery of high local concentration of the therapeutics to the adventitia, media, and intima [44].

Systemic administration of estradiol, i.e., E2 (Molecular Weight: 272.38 Formula: C18H24O Dissolves in absolute Ethanol) requires weekly therapeutic injections ([Study 1:10 weeks old+OVX (ovariectomy)+1 week+NX (nephrectomy)+4 weeks+AVF+4 weeks (with systemic E2 [100 ug/day, i.p])+4 weeks]) that are not applicable in real-life clinical settings. However, systemically delivered estrogen inhibits neointima formation in the venous arms of AV fistulae in animals with experimentally induced chronic kidney disease (CKD). The nanoparticle membrane disclosed herein may locally deliver nanoparticles for the carotid artery ligation model and potentially for the AV Fistula injury site, replacing weekly injections, without the requirement for elevated systemic levels of E2. Nanoparticles may be successfully delivered and retained in layers of carotid artery tissue on a mouse model with significant enhancement.

However, the initial nanoparticle formulation release profile was limited to one week. The nanoparticle release profile and loading efficiency was optimized using four different nanoparticle formulations via an experimental model. Optimal nanoparticle conditions were obtained with high loading efficiency and a 50-day sustained release profile of E2 (see FIGS. 4 and 6). Nanoparticles were prepared by a single emulsion method, and they were characterized for the drug content utilizing enzyme-linked immunosorbent assay (ELISA), release profile, size, surface morphology utilizing a scanning electron microscope (SEM), etc. Synthesized PLGA nanoparticles loaded with E2 were developed and incorporated onto a PLGA film platform. This novel combined therapy approach can overcome the challenges due to the irregular morphology of AV fistula injury sites. The film coated with E2 nanoparticles can easily wrap the injury site without requiring a suture and help E2 nanoparticles be absorbed into layers of vascular tissue.

Synthesized PLGA nanoparticles may be loaded with E2 and incorporated onto a PLGA film platform.

The E2-loaded nanoparticles coated onto the bio-degradable polymeric film prevent neointimal thickening. Thus, it reduces the time between surgery and when the fistula can be cannulated successfully for dialysis and minimizes the need for additional, supplementary interventions to help maintain fistula functionality.

Central to the present subject matter is the creation of drug-eluting film which can be used to administer a combinatorial cocktail of therapeutics that modulate multiple pathways that lead to AV fistula failure. This novel combined film/nanoparticle system may deliver enough E2 to the AV fistula injury site over time to reduce neointima formation while preserving normal serum E2 levels, thus enhancing the probability that the approach can be translated to the clinical level for patients (even male patients) with end-stage renal disease. Additionally, sustained release of E2 from the nanoparticles may maintain required E2 plasma levels, avoiding excessive administrations and thus eliminating E2 side effects.

A preferred gene therapy model is bi-layered stent coated with PLGA nanoparticles with Vascular Endothelial Growth Factor (VEGF) plasmid in the outer layer and any anti-proliferative therapeutic agents such as paclitaxel and/or microRNAs such as mRNA-21 in the inner core for the neointimal formation.

The key point for each of these therapies is to inhibit the growth of vascular SMCs while promoting re-endothelization of the vessel to reverse the injured vessel back into a healthy one. Prior art stents containing antiproliferative drugs that inhibit SMC and re-endothelization may be used to at first suppress SMC and endothelization since the body's first reaction to the inflammation promotes inflammation and re-endothelization with the body's natural VEGF gene. Both may be suppressed at first, and later the re-endothelization to heal the tissue itself may be promoted.

The product may be wrapped upon anastomosis and stay there 10-20 min long to make sure the nanoparticles are transferred from a poly(lactic-co-glycolic acid) (PLGA)-based membrane to targeted tissue, discard the membrane, and close the surgery site.

Most of the time, the human body reacts to foreign substances and accelerates the inflammation process, which results in the tissue taking longer to heal than expected. However, with this approach, the body's inflammation/ rejection process is prevented or minimized to the membrane itself.

This novel approach may deliver enough E2 or anti-miR-21 NPs to the AV fistula injury site over time to reduce neointima formation while preserving normal serum E2 levels. Additionally, sustained release of E2 from the nanoparticles would maintain required E2 plasma levels, avoiding excessive administrations and thus eliminating E2 side effects.

During the AV fistulae surgery, a surgeon applies the nanocarrier-coated film on the outside of a vessel or artery conveniently wrapping the injury site without requiring an extra suture. After a while, the body absorbs the film coated with E2 loaded nanoparticles into vascular tissue layers. The surgeon may close the surgery site. Estrogen's anti-inflammatory effect [45] helps the surgical scar of AV fistulae heal itself faster too.

### EXAMPLES

To cast polymeric sheaths, 220 mg of polymer (PLGA or PCL) was dissolved in about 2 to 2.2 ml of Dichloromethane (DCM). The polymer-solvent solution was cast into a 100 mm×100 mm polytetrafluoroethylene (PTFE) dish and kept in a fume hood for 48 h to evaporate the solvent. The casted films were cut into 5.0 cm×2.5 cm sheets and subsequently vacuumed dried overnight in the dark to eliminate residual solvent. Polymeric sheaths were stored at −20 Celsius until use.

Ticlopidine (1800 mg) may be added to the polymer solution prior to casting and stirred for 30 minutes in the dark. Each 5.0 cm×2.5 cm sheet is estimated to contain about 230 mg ticlopidine.

Anti-miR-21 NPs in a paste form are applied onto the ticlopidine eluting film. The coated film is longitudinally placed onto an injured segment (about 5 cm) of a common carotid artery and wrapped in such a way that the sheath covers about 90% of the outer surface of the vessel. See FIG. 14.

PLGA-based Estrogen Nanoparticles were prepared and characterized as follows. PLGA (lactide-to-glycolide molar ratio: 50:50, molecular weight: 7,000-17,000) and polyvinyl alcohol (PVA); molecular weight: 89,000-98,000) were purchased from Fisher Scientific. PLGA nanoparticles were prepared via a double emulsion (water/oil/water phase) technique. A solution of PLGA (100 mg) in dichloromethane (3 ml) and 2 ml estrogen (17 β-estradiol; E2) (30 mg/ml in absolute Ethanol) was ultrasonicated at 40% amplitude in about 40-s intervals with 20-s pauses for a total of 2 min; then 20 ml of a 4% (wt/vol) PVA-water solution was added, and the mixture was ultrasonicated on ice at 40% amplitude in ˜40-s intervals with 20-s pauses for a total of 2 min. The mixture was transferred with 10 ml of a 4% (wt/vol) PVA-water solution and 30 ml of Milli-Q® water to a 100-ml glass beaker and stirred for four hours until the dichloromethane evaporated. The solution was then centrifuged at 1,000 G for 10 min to remove any aggregates, and the supernatant was removed and centrifuged at 45,000 G for 20 min to collect the nanoparticles. The nanoparticles were washed three times (to remove any PVA) by resuspending them in 50 ml of Milli-Q® water and recollecting them via centrifugation at 45,000 G for 20 min. The nanoparticles were then frozen at −80° C. overnight, lyophilized for 48 h, and stored in Eppendorf tubes at −80° C.

PLGA-based anti-miRNA-21 nanoparticles are prepared as discussed below: PLGA nanoparticles were prepared via our modified double emulsion (water/oil/water phase) technique. Briefly, a solution of PLGA (100 mg) in dichloromethane (5 ml) with or without miRNA content (200 μl at 5 nmol) and with or without coumarin-6 (1 mg) was ultrasonicated at 40% amplitude in 40-s intervals with 20-s pauses for a total of 2 min; 20 ml of a 4% (wt/vol) PVA-water solution was then added, and the mixture was ultrasonicated on ice at 40% amplitude in ˜40-s intervals with 20-s pauses for a total of 2 min. The mixture was transferred to 10 ml of a 4% (wt/vol) PVA-water solution and 30 ml of Milli-Q® water to a 100-ml glass beaker and stirred for 4 h until the dichloromethane evaporated. The solution was then centrifuged at 1,000 G for 10 min to remove any aggregates, and the supernatant was removed and centrifuged at 45,000 G for 20 min to collect the nanoparticles. The nanoparticles were washed three times (to remove any PVA) by resuspending them in 50 ml of Milli-Q® water and recollecting them via centrifugation at 45,000 G for 20 min and then frozen at −80° C. overnight, lyophilized for 48 h, and stored in Eppendorf tubes at −80° C.

To prevent and treat neointima formation according to another embodiment of the present invention, Matrigel® solubilized basement membrane matrix was mixed with a 10 mg/ml suspension of estrogen nanoparticles (suspended in Millipore® water) at a one-to-one ratio by ultra-sonication. The Matrigel® +Estrogen NP mix was kept on ice during surgery. 200 ml of the mix was pipetted onto an AV Fistulae surgery site. After ten minutes, the solidified gel nicely wrapped the vein and the artery. Then the skin of the rat was sutured. The gel was checked 1-day and 5-days after surgery to confirm the gel was intact with the tissue.

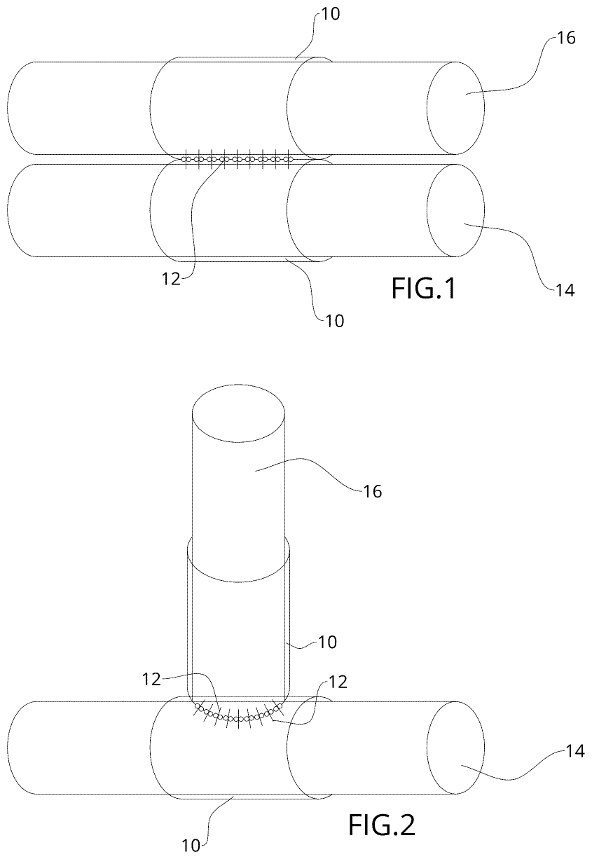

Referring to FIGS. 1 through 9, FIGS. 1 and 2 illustrate nanoparticle-loaded films 10 according to an embodiment of the present invention wrapped around an artery 14 and a vein 16 at a surgical site having sutures 12. Note that the films 10 do not need any suture to stabilize the product on the site. The films 10 shown are ticlopidine eluting PLGA film with anti-miR-21 nanoparticles thereon, applied perivascularly on a radiocephalic AV fistula and/or a basilic vein AV fistula and/or a brachiocephalic AV fistula. FIG. 1 shows a side-to-side anastomosis, while FIG. 2 shows an end-to-side anastomosis.

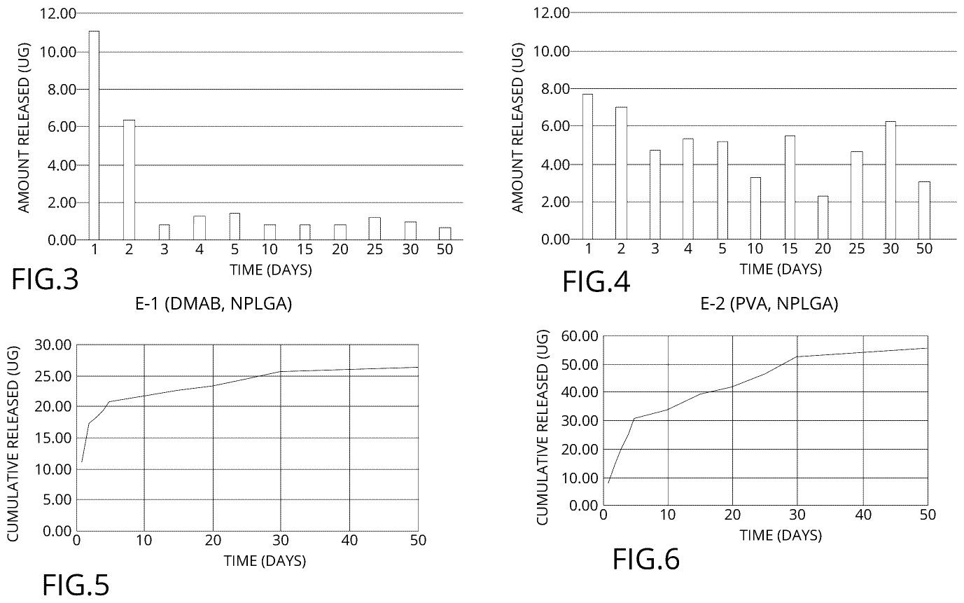

FIG. 3 illustrates an amount of estrogen-1 (E-1) released from PLGA nanoparticles (nPLGA) over a period of 50 days in didodecyl dimethyl ammonium bromide (DMAB). FIG. 5 illustrates the cumulative amount released.

FIG. 4 illustrates an amount of estrogen-2 (E-2) released from PLGA nanoparticles (nPLGA) over a period of 50 days in polyvinyl alcohol (PVA). FIG. 6 illustrates the cumulative amount released.

E2-loaded nanoparticles absorb into vascular tissue layers. An enhanced nanoparticle release profile and loading efficiency may be used—the optimal nanoparticle conditions obtained with high loading efficiency and a 50-day sustained release profile of E2.

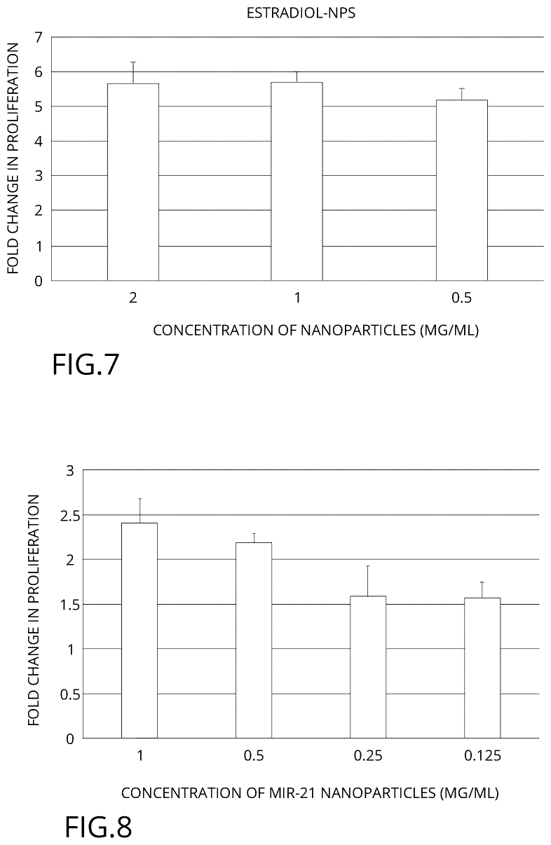

FIGS. 7 and 8 display the results of colorimetric match to sample (MTS) assays illustrating a change in human smooth muscle cell (SMC) proliferation over 48 hours as a function of nanoparticle concentration. MTS assay may also be used to measure cytotoxicity to predict any in vitro toxic effect due to the nanoparticles. Induced Pluripotent Stem Cells (IPS) differentiated into SMCs, which were treated with estradiol nanoparticles for 48 hours. FIG. 7 illustrates the effect of estradiol nanoparticles, whereas FIG. 8 illustrates the effect of miR-21 nanoparticles. The Y axis title “fold change in proliferation” refers to nanoparticle treated cells divided by cells with only medium but no treatment. Naked miRNAs are not stable in circulating blood. Thus, the therapeutic effect of miR-21 might not be induced by naked miR-21 [46], [25]. So, the present examples used mimic microRNAs that are encapsulated in PLGA nanoparticles via a modified double emulsion method (see FIG. 16).

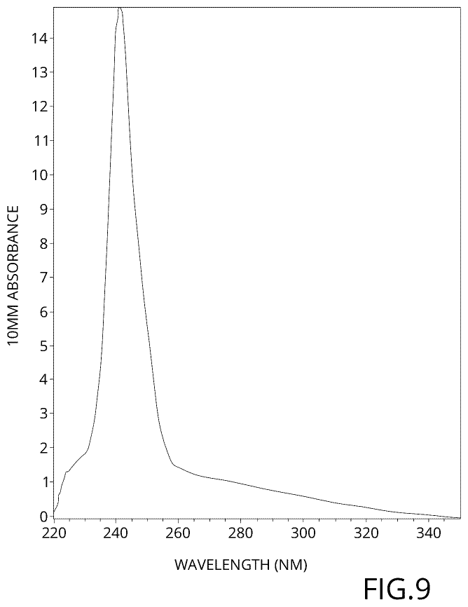

Nanocarriers according to an embodiment of the present invention were analyzed as follows. Nanoparticles were dissolved to extract miR-21 content and the amount of miR-21 content encapsulated was measured (particle loading and in vitro release) via nanodrop spectrophotometer (see FIG. 9), enabling optimization of formulations. PLGA nanoparticles (FIG. 16) with approximate mean diameters of 150 nm were generated by a single emulsion technique; the encapsulation efficiency (i.e., the amount encapsulated/total amount available×100%) was 91%, and the concentration of encapsulated microRNA was 264.15 ng microRNA content/mg nanoparticles (FIG. 9). When 1 mg of microRNA 21-loaded nanoparticles was incubated in 1 ml phosphate buffered saline (PBS) at 37° C., 10% of the encapsulated microRNA 21 was released during the 24 hours, 13% was released by day 10 and 19% was released by day 14.

FIG. 9 is a content profile for miR-21 nanoparticles, showing an absorbance peak at a wavelength of about 240 nm.

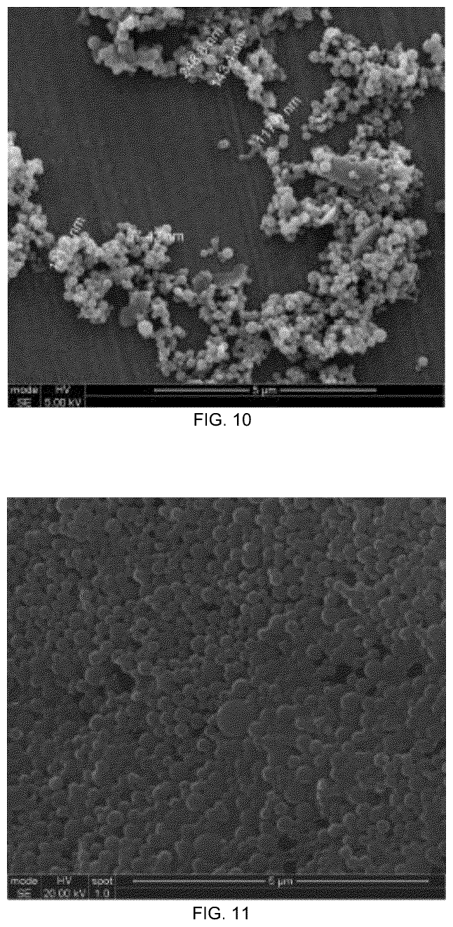

FIGS. 10 and 11 show estrogen-2 loaded nanoparticles under a microscope. FIG. 10 shows sizes at 100 nm. FIG. 11 shows sizes at 192 nm. Anti-miR-21 in the size range of 100-200 nanometer have low immunogenic, spherical, and smooth surfaces, with more monodisperse and stable particles[47].

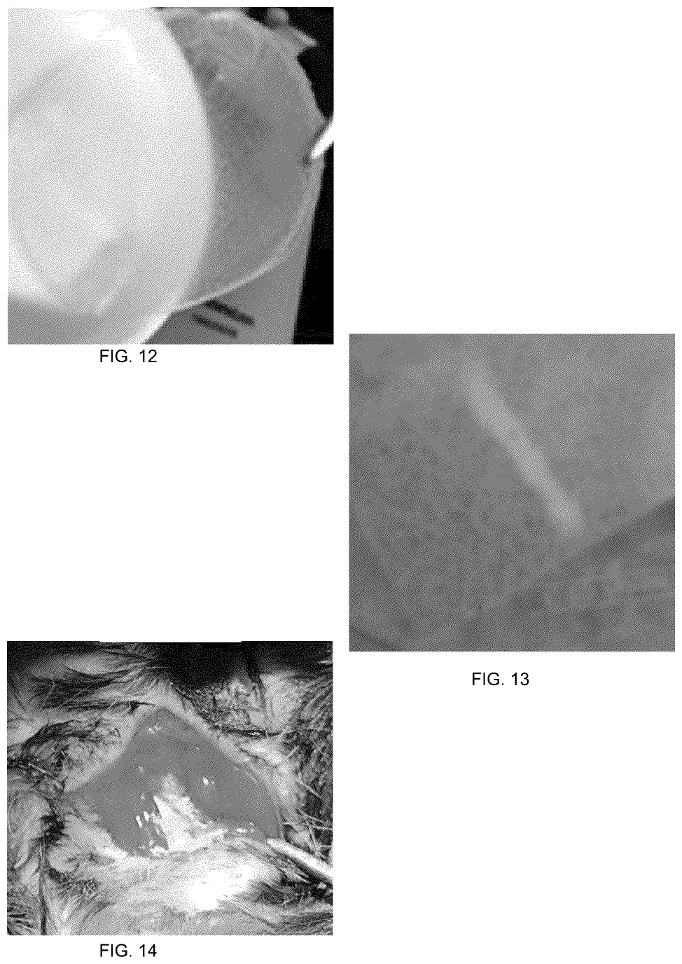

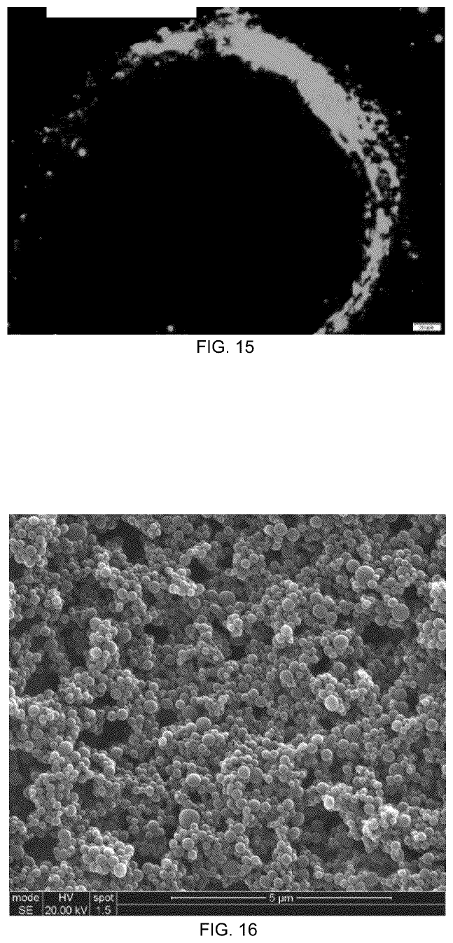

FIG. 12 is a photographic view of a PLGA film without nanoparticles. FIG. 13 is a photographic view of a drug-eluting nanofilm loaded with coumarin-6 nanoparticles, shown as a light-colored diagonal streak. FIG. 14 illustrates the film of FIG. 13 applied to injured mouse carotid artery tissue. The coumarin-6 nanoparticles are shown as a light-colored region at the center of the photograph. FIG. 15 is a microscopic image of the coumarin-6 nanoparticle-containing film of FIG. 14 one day after surgery.

FIG. 16 is a SEM image of microRNA-21 nanoparticle content.

It should be understood, of course, that the foregoing relates to exemplary embodiments of the invention and that modifications may be made without departing from the spirit and scope of the invention as set forth in the following claims.

### REFERENCES

[1] M. Thamer et al., “Medicare Costs Associated With Arteriovenous Fistulas Among US Hemodialysis Patients,” Am. J. Kidney Dis., vol. 72, no. 1, pp. 10-18, Jul. 2018, doi: 10.1053/J.AJKD.2018.01.034/ATTACHMENT/C897FDEF-B625-4085-9624-913824B63D07/MMC5.PDF.
[2] “Annual Data Report|USRDS.” https://adr.usrds.org/2020/ (accessed Feb. 27, 2022).
[3] A. Asif, P. Roy-Chaudhury, and G. A. Beathard, “Early Arteriovenous Fistula Failure: A Logical Proposal for When and How to Intervene,” Clin. J. Am. Soc. Nephrol., vol. 1, no. 2, pp. 332-339, March 2006, doi: 10.2215/CJN.00850805.
[4] J. M. MacRae et al., “Arteriovenous Access Failure, Stenosis, and Thrombosis,” Can. J. Kidney Heal. Dis., vol. 3, no. 1, 2016, doi: 10.1177/2054358116669126.
[5] E. A. Takahashi, W. S. Harmsen, and S. Misra, “Endovascular Arteriovenous Dialysis Fistula Intervention: Outcomes and Factors Contributing to Fistula Failure,” Kidney Med., vol. 2, no. 3, pp. 326-331, May 2020, doi: 10.1016/J.XKME.2020.02.004.
[6] L. Denes, L. Entz, and V. Jancsik, “Restenosis and Therapy,” Int. J. Vasc. Med., vol. 2012, 2012, doi: 10.1155/2012/406236.
[7] S. G. Achinger and J. C. Ayus, “Inflammation from dialysis, can it be removed?,” Nephrol. Dial. Transplant, vol. 28, no. 4, pp. 770-773, April 2013, doi: 10.1093/NDT/GFS480.
[8] M. Alton et al., “Medial fibrosis, vascular calcification, intimal hyperplasia, and arteriovenous fistula maturation,” Am. J. Kidney Dis., vol. 58, no. 3, pp. 437-443, September 2011, doi: 10.1053/J.AJKD.2011.04.018.
[9] M. Allan et al., “Preoperative venous intimal hyperplasia, postoperative arteriovenous fistula stenosis, and clinical fistula outcomes,” Clin. J. Am. Soc. Nephrol., vol. 8, no. 10, pp. 1750-1755, October 2013, doi: 10.2215/CJN.02740313.
[10] M. Allan et al., “Correlation of Pre-existing Vascular Pathology With Arteriovenous Graft Outcomes in Hemodialysis Patients,” Am. J. Kidney Dis., vol. 62, no. 6, pp. 1122-1129, December 2013, doi: 10.1053/J.AJKD.2013.03.040.
[11] M. Allan et al., “Preoperative Arterial Microcalcification and Clinical Outcomes of Arteriovenous Fistulas for Hemodialysis,” Am. J. Kidney Dis., vol. 66, no. 1, p. 84, July 2015, doi: 10.1053/J.AJKD.2014.12.015.
[12] Y. 0. Kim et al., “Preexisting intimal hyperplasia of radial artery is associated withearly failure of radiocephalic arteriovenous fistula in hemodialysis patients,” Am. J. Kidney Dis., vol. 41, no. 2, pp. 422-428, Feb. 2003, doi: 10.1053/ajkd.2003.50051.
[13] “Neointimal Hyperplasia PubMed.”

https://pubmed.ncbi.nlm.nih.gov/29763068/ (accessed Mar. 2, 2022).
[14] E. Ban, T. H. Kwon, and A. Kim, “Delivery of therapeutic miRNA using polymer-based formulation,” Drug Deliv. Transl. Res., vol. 9, no. 6, pp. 1043-1056, December 2019, doi: 10.1007/S13346-019-00645-Y.
[15] T. J. Sill and H. a von Recum, “Electrospinning: applications in drug delivery and tissue engineering.,” Biomaterials, vol. 29, no. 13, pp. 1989-2006, May 2008, doi: 10.1016/j.biomaterials.2008.01.011.
[16] Y. S. Nam and T. G. Park, “Protein loaded biodegradable microspheres based on PLGA-protein bioconjugates,” J. Microencapsul., vol. 16, no. 5, pp. 625-637, January 1999, doi: 10.1080/026520499288816.
[17] E. Schnell et al., “Guidance of glial cell migration and axonal growth on electrospun nanofibers of poly-[epsilon]-caprolactone and a collagen/poly-[epsilon]-caprolactone blend,” Biomaterials, vol. 28, no. 19, pp. 3012-3025, July 2007, doi: 10.1016/j.biomaterials.2007.03.009.
[18] R. C. Lee, R. L. Feinbaum, and V. Ambros, “The C. elegans heterochronic gene lin-4 encodes small RNAs with antisense complementarity to lin-14,” Cell, vol. 75, no. 5, pp. 843-854, Dec. 1993, doi: 10.1016/0092-8674(93)90529-Y.
[19] A. E. Pasquinelli, S. Hunter, and J. Bracht, “MicroRNAs: a developing story,” Curr. Opin. Genet. Dev., vol. 15, no. 2, pp. 200-205, 2005, doi: 10.1016/J.GDE.2005.01.002.
[20] B. P. Lewis, C. B. Burge, and D. P. Bartel, “Conserved seed pairing, often flanked by adenosines, indicates that thousands of human genes are microRNA targets,” Cell, vol. 120, no. 1, pp. 15-20, January 2005, doi: 10.1016/J.CELL.2004.12.035.
[21] R. Ji et al.,“MicroRNA expression signature and antisense-mediated depletion reveal an essential role of MicroRNA in vascular neointimal lesion formation,” Circ. Res., vol. 100, no. 11, pp. 1579-1588, June 2007, doi: 10.1161/CIRCRESAHA.106.141986.
[22] “microRNA Market Size, Share|Industry Report, 2021-2028.” https://www.grandviewresearch.com/industry-analysis/microrna-market (accessed Apr. 23, 2022).
[23] C. C. Wu et al., “MicroRNA-21 and venous neointimal hyperplasia of dialysis vascular access,” Clin. J. Am. Soc. Nephroi., vol. 13, no. 11, pp. 1712-1720, 2018, doi: 10.2215/CJN.02410218/-/DCSUPPLEMENTAL.
[24] R. Ji et al.,“MicroRNA expression signature and antisense-mediated depletion reveal an essential role of MicroRNA in vascular neointimal lesion formation,” Circ. Res., vol. 100, no. 11, pp. 1579-1588, June 2007, doi: 10.1161/CIRCRESAHA.106.141986.
[25] Y. Cheng and C. Zhang, “MicroRNA-21 in Cardiovascular Disease,” J. Cardiovasc. Transl. Res., vol. 3, no. 3, p. 251, June 2010, doi: 10.1007/S12265-010-9169-7.
[26] R. Ji et al., “MicroRNA expression signature and antisense-mediated depletion reveal an essential role of MicroRNA in vascular neointimal lesion formation.,” Circ. Res., vol. 100, no. 11, pp. 1579-88, June 2007, doi: 10.1161/CIRCRESAHA.106.141986.
[27] J. Gorecka et al., “Molecular targets for improving arteriovenous fistula maturation and patency,” Vasc. Investig. Ther., vol. 2, no. 2, p. 33, 2019, doi: 10.4103/vit.vit_9_19.
[28] K. Wolf, H. Hu, T. Isaji, and A. Dardik, “Molecular identity of arteries, veins, and lymphatics,” J. Vasc. Surg., vol. 69, no. 1, pp. 253-262, January 2019, doi: 10.1016/J.JVS.2018.06.195.
[29] R. S. Ashton et al., “Astrocytes regulate adult hippocampal neurogenesis through ephrin-B signaling,” Nat. Neurosci., vol. 15, no. 10, pp. 1399-1406, October 2012, doi: 10.1038/NN.3212.
[30] J. J. Steinle, C. J. Meininger, R. Forough, G. Wu, M. H. Wu, and H. J. Granger, “Eph B4 receptor signaling mediates endothelial cell migration and proliferation via the phosphatidylinositol 3-kinase pathway,” J. Biol. Chem., vol. 277, no. 46, pp. 43830-43835, November 2002, doi: 10.1074/JBC.M207221200.
[31] C. D. Protack et al., “Eph-B4 regulates adaptive venous remodeling to improve arteriovenous fistula patency,” Sci. Rep., vol. 7, no. 1, December 2017, doi: 10.1038/S41598-017-13071-2.
[32] A. Ghorbani and F. Jasemi-Zergani, “Ticlopidine to prevent primary arteriovenous fistula failure in hemodialysis patients; a randomized controlled trial,” J. Ren. Inj. Prev., vol. 2, no. 3, p. 109, 2013, doi: 10.12861/JRIP.2013.35.
[33] M. J. Quinn and D. J. Fitzgerald, “Ticlopidine and Clopidogrel,” Circulation, vol. 100, no. 15, pp. 1667-1672, October 1999, doi: 10.1161/01.CIR.100.15.1667.
[34] M. De Lorgeril et al., “Ticlopidine increases nitric oxide generation in heart-transplant recipients: a possible novel property of ticlopidine,” J. Cardiovasc. Pharmacol., vol. 32, no. 2, pp. 225-230, August 1998, doi: 10.1097/00005344-199808000-00009.
[35] M. R. Bowling et al., “Estrogen Effects on Vascular Inflammation Are Age Dependent: Role of Estrogen Receptors,” Arterioscler. Thromb. Vasc. Biol., vol. 34, no. 7, pp. 1477-1485, July 2014, doi: 10.1161/ATVBAHA.114.303629.
[36] G. Li, Y. F. Chen, G. L. Greene, S. Oparil, and J. A. Thompson, “Estrogen inhibits vascular smooth muscle cell-dependent adventitial fibroblast migration in vitro.,” Circulation, vol. 100, no. 15, pp. 1639-45, October 1999, doi: 10.1161/01.cir.100.15.1639.
[37] W. A. Dorsett-Martin and R. L. Hester, “Sex hormones and aortic wall remodeling in an arteriovenous fistula,” Gend. Med., vol. 4, no. 2, pp. 157-169, June 2007, doi: 10.1016/S1550-8579(07)80029-5.
[38] A. Abizaid et al., “First human experience with the 17-beta-estradiol-eluting stent: The estrogen and stents to eliminate restenosis (EASTER) trial,” J. Am. Coll. Cardiol., vol. 43, no. 6, pp. 1118-1121, 2004, doi: 10.1016/j.jacc.2004.01.023.
[39] A. M. Deschamps, E. Murphy, and J. Sun, “Estrogen Receptor Activation and Cardioprotection in Ischemia Reperfusion Injury,” Trends Cardiovasc. Med., vol. 20, no. 3, pp. 73-78, April 2010, doi: 10.1016/J.TCM.2010.05.001.
[40] Y. D. Krom et al., “Inhibition of neointima formation by local delivery of estrogen receptor alpha and beta specific agonists,” Cardiovasc. Res., vol. 73, no. 1, pp. 217-226, January 2007, doi: 10.1016/J.CARDIORES.2006.10.024.
[41] C. R. White et al., “Estrogen Restores Endothelial Cell Function in an Experimental Model of Vascular Injury,” Circulation, vol. 96, no. 5, pp. 1624-1630, September 1997, doi: 10.1161/01.CIR.96.5.1624.
[42] “Local application of calcium antagonists inhibits intimal hyperplasia after arterial injury—PubMed.” https://pubmed.ncbi.nlm.nih.gov/8121548/(accessed Jan. 1, 2023).
[43] A. Chaux et al., “Perivascular delivery of a nitric oxide donor inhibits neointimal hyperplasia in vein grafts implanted in the arterial circulation,” J. Thorac. Cardiovasc. Surg., vol. 115, no. 3, pp. 604-614, 1998, doi: 10.1016/S0022-5223(98)70325-3.
[44] K. B et al., “Perivascular paclitaxel wraps block arteriovenous graft stenosis in a pig model,” Nephrol. Dial. Transplant, vol. 21, no. 9, pp. 2425-2431, September 2006, doi: 10.1093/NDT/GFL250.
[45] A. Villa, N. Rizzi, E. Vegeto, P. Ciana, and A. Maggi, “Estrogen accelerates the resolution of inflammation in macrophagic cells,” Sci. Reports 2015 51, vol. 5, no. 1, pp. 1-14, Oct. 2015, doi: 10.1038/srep15224.
[46] J. Kota et al., “Therapeutic microRNA delivery suppresses tumorigenesis in a murine liver cancer model,” Cell, vol. 137, no. 6, pp. 1005-1017, June 2009, doi: 10.1016/J.CELL.2009.04.021.
[47] S. Xiong et al., “Size influences the cytotoxicity of poly (lactic-co-glycolic acid) (PLGA) and titanium dioxide (TiO2) nanoparticles,” Arch. Toxicol., vol. 87, no. 6, p. 1075, June 2013, doi: 10.1007/S00204-012-0938-8.

What is claimed is:

1. An implantable drug delivery device, comprising:
a biodegradable substrate and
polymeric nanoparticles containing a therapeutic agent affixed to the biodegradable substrate.

2. The implantable drug delivery device of claim 1, wherein the polymeric nanoparticles comprise a polymer selected from the group consisting of: poly(lactic-co-glycolic acid), polycaprolactone, and a poloxamer.

3. The implantable drug delivery device of claim 1, wherein the polymeric nanoparticles are conjugated with a tissue-specific ligand.

4. The implantable drug delivery device of claim 1, wherein the biodegradable substrate contains a platelet aggregation inhibitor.

5. The implantable drug delivery device of claim 1, wherein the biodegradable substrate is a polymeric film, a polymeric stent, a perivascular sheath, or a gel.

6. The implantable drug delivery device of claim 1, wherein the biodegradable substrate comprises poly(lactic-co-glycolic acid), polycaprolactone, or a basement membrane matrix.

7. The implantable drug delivery device of claim 6, wherein the polymeric stent has an inner layer of antiproliferative agent and/or microRNA coated with a vascular endothelial growth factor plasmid.

8. The implantable drug delivery device of claim 1, wherein the therapeutic agent is selected from the group consisting of microRNA, an estrogen, and a combination thereof.

9. The implantable drug delivery device of claim 8, wherein the microRNA is selected from the group consisting of: microRNA-21, anti-microRNA-21, and a microRNA-21 oligonucleotide.

10. The implantable drug delivery device of claim 8, wherein the estrogen is estradiol.

11. A method of preventing or reversing vascular hemodialysis access dysfunction, comprising:
wrapping an anastomosis injury site with the implantable drug delivery device of claim 1.

12. The method of claim 11, wherein the biodegradable substrate is not sutured to the anastomosis injury site.

13. The method of claim 11, further comprising discarding the biodegradable substrate after a predetermined time; and closing the anastomosis injury site.

14. The method of claim 11, further comprising delivering an elevated local concentration of the therapeutic agent to a vessel component at the anastomosis injury site selected from the group consisting of: an adventitia, a media, and an intima.

15. A method of making the implantable drug delivery device of claim 1, comprising:
forming the biodegradable substrate;
forming the polymeric nanoparticles containing the therapeutic agent by encapsulation and/or from an emulsion; and
applying the polymeric nanoparticles to the biodegradable substrate.

16. The method of claim 15, wherein the polymeric nanoparticles are present as a paste.
